(19) United States
(12) Patent Application Publication
(10) Pub. No.: US 2011/0201568 A1
(43) Pub. Date: Aug. 18, 2011

(54) PHYOTOCHEMICAL COMPOSITION AND A PROCESS THEREOF

(76) Inventor: Amit Agarwal, Bangalore (IN)

(21) Appl. No.: 13/125,531

(22) PCT Filed: Nov. 25, 2009
(86) PCT No.: PCT/IN2009/000682

§ 371 Date: Apr. 21, 2011

(30) Foreign Application Priority Data
Nov. 26, 2008 (IN) 02957/CHE/2008

(51) Int. Cl. A61K 31/704; A61K 31/235; A61P 1/04

(52) U.S. Cl. 514/33; 514/533

(57) ABSTRACT

The present invention is successful in providing caffeic acid derivatives namely eicosanyl caffeate and docosyl caffeate from Glycyrrhiza. Also, the present invention provides a composition comprising said bio actives and also provides a process to prepare the composition. Ultimately, the invention provides solution for the management of ulcer.

### FIELD OF THE INVENTION

The present invention relates to phytochemical composition for management of ulcer. More particularly, the present invention provides a composition, a process to prepare the composition from Glycyrrhiza species for management of ulcer, gut mucosal health and gut related disorders.

### BACKGROUND AND PRIOR ART OF THE INVENTION

The prevalence of gastric mucosal damage due to hyperacidity, ulcer, use of nonsteroidal anti-inflammatory drugs and alcohol consumption is rapidly increasing. Among other factors (like Helicobacter pylori), neutrophil derived elastase and oxidative stress resulting from oxygen derived free radicals like superoxide anion (O₂.) and hydroxyl (OH⁻) radicals are considered to significantly contribute to the gastric damage [W. Liu, K. Okajima, K. Murakami, N. Harada, H. Isobe, T. Irie J. Lab. Clin. Med., 132(5), 432 (1998). M. G. Repetto, S. F. Llesny Braz. J. Med. Biol. Res., 35, 523 (2002).] It is therefore considered desirable that gastroprotective agents possess elastase inhibitory and antioxidant activity.

Prevalence: Researchers recognize three major causes of peptic ulcer disease: infection with Helicobacter pylori (formerly known as Campylobacter pylori), use of NSAIDs, and pathologic hypersecretory disorders such as Zollinger-Ellison syndrome. How H. pylori produces an ulcer isn't clear. Gastric acid, which was considered a primary cause, now appears mainly to contribute to the consequences of infection.

H. pylori Infection and Oxidative Stress:

Increased oxidative stress represents an important mechanism leading to epithelial injury in H. pylori infection. Both bacterial and host factors contribute to the oxidative stress induced by infection.

Various Factors Responsible for Induction of Oxidative Stress in Gastric Epithelial Cells in H. pylori Infection:
√ Generation of reactive oxygen species (ROS) by H. pylori.
√ Recruitment of phagocytes and pro-inflammatory cytokines.
√ Decreased levels of ascorbic acid.

Ongoing studies should soon unveil the full mechanism of ulcer formation. Salicylates and other NSAIDs encourage ulcer formation by inhibiting the secretion of prostaglandins (the substances that suppress ulceration). Certain illnesses, such as pancreatitis, hepatic disease, Crohn's disease, preexisting gastritis, and Zollinger-Ellison syndrome, are also known causes.

Besides peptic ulcer's main causes, several predisposing factors are acknowledged. They include blood type (gastric ulcers tend to strike people with type A blood; duodenal ulcers tend to afflict people with type O blood) and other genetic factors. Exposure to irritants, such as alcohol, coffee, and tobacco, may contribute by accelerating gastric acid emptying and promoting mucosal breakdown. Ulceration occurs when the acid secretion exceeds the buffering factors. Physical trauma, emotional stress, and normal aging are additional predisposing conditions.

In the United States, about 1.6 million people acquire peptic ulcers yearly. Males and females are affected equally, and incidence increases with age. A higher percentage of H. pylori infection occurs in people older than age 50. Probably it is considered to be most common chronic infection of humans. Human populations throughout the world affected. Incidence increases with age and occurs earlier and at increased rates in the developing world and lower socioeconomic groups. Up to 90% of some populations infected.

### The Following Statistics Relate to the Prevalence of Peptic Ulcer:

22 per 1000 (NHIS95)
2.7% of female population self-reported having stomach, duodenal or gastrointestinal ulcers in Australia 2001 (ABS 2001 National Health Survey, Australia's Health 2004, AIHW)
2.8% of male population self-reported having stomach, duodenal or gastrointestinal ulcers in Australia 2001 (ABS 2001 National Health Survey, Australia's Health 2004, AIHW)
249,000 men self-reported having stomach, duodenal or gastrointestinal ulcers in Australia 2001 (ABS 2001 National Health Survey, Australia's Health 2004, AIHW)
256,000 women self-reported having stomach, duodenal or gastrointestinal ulcers in Australia 2001 (ABS 2001 National Health Survey, Australia's Health 2004, AIHW)
506,000 people self-reported having stomach, duodenal or gastrointestinal ulcers in Australia 2001 (ABS 2001 National Health Survey, Australia's Health 2004, AIHW)
   Source: www.wrongdiagnosis.com/p/peptic_ulcer/prevalence.htm—43 k—

Current management strategies: Because many ulcers stem from H. pylori bacteria, doctors use a two-pronged approach to peptic ulcer treatment:
Kill the bacteria.
Reduce the level of acid in your digestive system to relieve pain and encourage healing.

Accomplishing these two goals requires the use of at least two, and sometimes three or four, of the following medications:

Antibiotic medications: Doctors use combinations of antibiotics to treat H. pylori because one antibiotic alone isn't sufficient to kill the organism. For the treatment to work, it's essential that to follow doctor's instructions precisely. Antibiotics commonly prescribed for treatment of H. pylori include amoxicillin, clarithromycin and metronidazole.

Acid blockers: Also called histamine (H-2) blockers—reduce the amount of hydrochloric acid released into the digestive tract, which relieves ulcer pain and encourages healing. Acid blockers work by keeping histamine from reaching histamine receptors. Histamine is a substance normally present in the body. When it reacts with histamine receptors, the receptors signal acid-secreting cells in the stomach to release hydrochloric acid. Available by prescription or over-the-counter (OTC), acid blockers include the medications ranitidine, famotidine, cimetidine and nizatidine.

Antacids: An antacid may be taken in addition to an acid blocker or in place of one. Instead of reducing acid secretion, antacids neutralize existing stomach acid and can provide rapid pain relief.

Proton pump inhibitors: Another way to reduce stomach acid is to shut down the “pumps” within acid-secreting cells. Proton pump inhibitors reduce acid by blocking the action of these tiny pumps. These drugs include the prescription medications omeprazole, lansoprazole, rabeprazole and esomeprazole. The drug pantoprozole can be taken orally or administered intravenously in the hospital. Proton pump inhibitors are frequently prescribed to promote the healing of peptic ulcers. Proton pump inhibitors also appear to inhibit H. pylori. However, long-term use of proton pump inhibitors, particularly at high doses, may increase the risk of hip fracture.

Cytoprotective agents: In some cases, physician may prescribe these medications that help protect the tissues that line the stomach and small intestine. They include the prescription medications sucralfate and misoprostol. Another nonprescription cytoprotective agent is bismuth subsalicylate. In addition to protecting the lining of the stomach and intestines, bismuth preparations appear to inhibit H. pylori activity.

If H. pylori is not identified in the system, then it's likely that the ulcer is due to NSAIDs—which should be avoided using, if possible—or acid reflux, which can cause esophageal ulcers. In both cases, physician tries to reduce acid levels—through use of acid blockers, antacids or proton pump inhibitors—and may also you use cytoprotective drugs.

### Demerits of Current Therapies

Side effects—Up to 50 percent of people have side effects of H. pylori treatment. Side effects are usually mild, with fewer than 10 percent of patients stopping treatment because of side effects. For those who do experience side effects, it may be possible to make adjustments in the dose or timing of medication. Some of the most common side effects are described below.
Some of the treatment regimens use a medication called metronidazole or clarithromycin. These medications can cause a metallic taste in the mouth.
Alcoholic beverages (eg, beer, wine) should be avoided while taking metronidazole; the combination can cause skin flushing, headache, nausea, vomiting, sweating and a rapid heart rate.
Bismuth, which is contained in some of the regimens, causes the stool to become black and may cause constipation.
Many of the regimens cause diarrhea and stomach cramps.
   Source: http://www.uptodate.com/patients/content/topic.do?topicKey=˜gVxhkoEDMbXE/v

### OBJECTIVES OF THE PRESENT INVENTION

The principle objective of the present invention is to prepare a phytochemical composition comprising eicosanyl caffeate and docosyl caffeate. Another objective of the present invention is to prepare a composition from Glycyrrhiza species

Yet another objective of the present invention is to develop a process for preparation phytochemical composition capable of elastase inhibition from Glycyrrhiza species. Still another objective of the present invention is to make use of phytochemical composition comprising eicosanyl caffeate and docosyl caffeate for management of ulcer.

### STATEMENT OF THE PRESENT INVENTION

Accordingly, the present invention is in relation to a phytochemical composition comprising eicosanyl caffeate and docosyl caffeate; a pharmaceutical composition comprising eicosanyl caffeate and docosyl caffeate optionally along with acceptable additives; a process for preparation of phytochemical composition comprising eicosanyl caffeate and docosyl caffeate, comprising steps of: extracting powdered plant parts with solvents to obtain extract; filtering, drying and fractionating the extract to obtain a residue; and subjecting the residue to preparative HPLC to obtain the phytochemical composition; and use of a phytochemical composition comprising eicosanyl caffeate and docosyl caffeate to manufacture a medicament for holistic management of ulcer, gut mucosal health and gut related disorders in a subject in need thereof.

### BRIEF DESCRIPTION OF THE ACCOMPANYING DRAWINGS

FIG. 1 Histogram showing volume of gastric content of treated groups in pylorus ligation induced ulcer model using albino wistar rats.

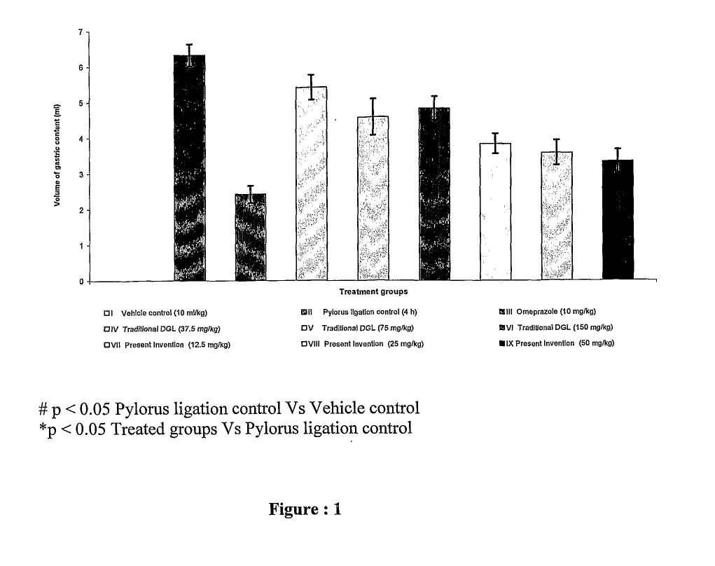

FIG. 2 Histogram showing pH values of treated groups in pylorus ligation induced ulcer model using albino wistar rats.

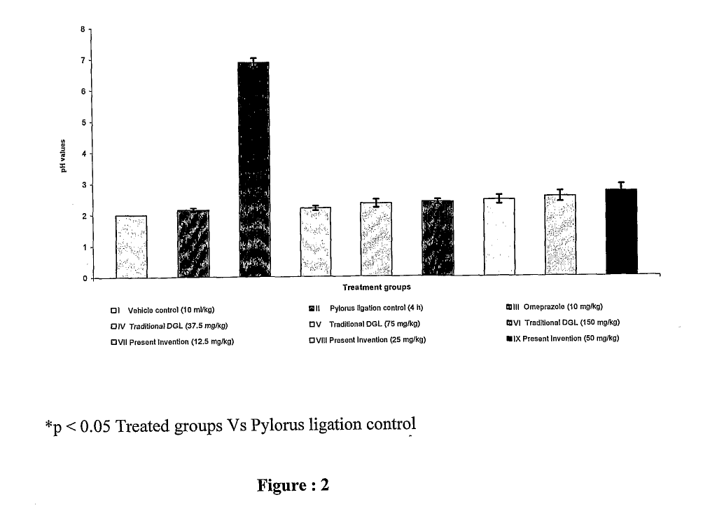

FIG. 3 Histogram showing total acidity of gastric juice of treated groups in pylorus ligation induced ulcer model using albino wistar rats.

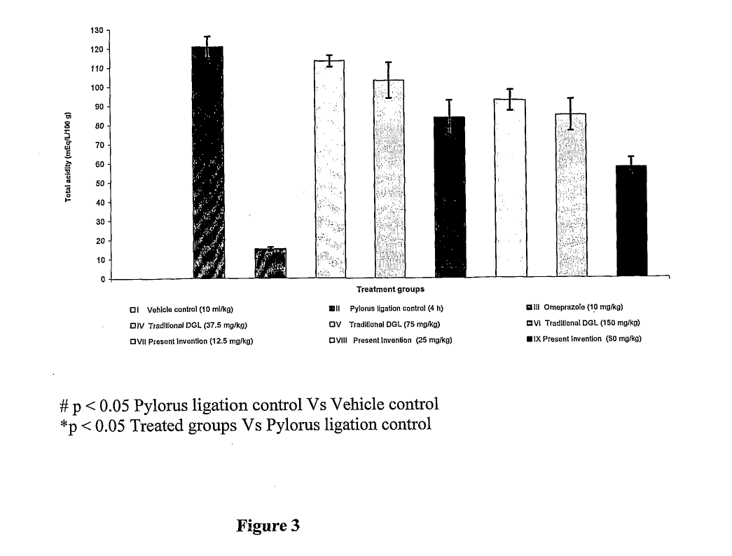

FIG. 4 Histogram showing ulcer index of treated groups in pylorus ligation induced ulcer model using albino wistar rats.

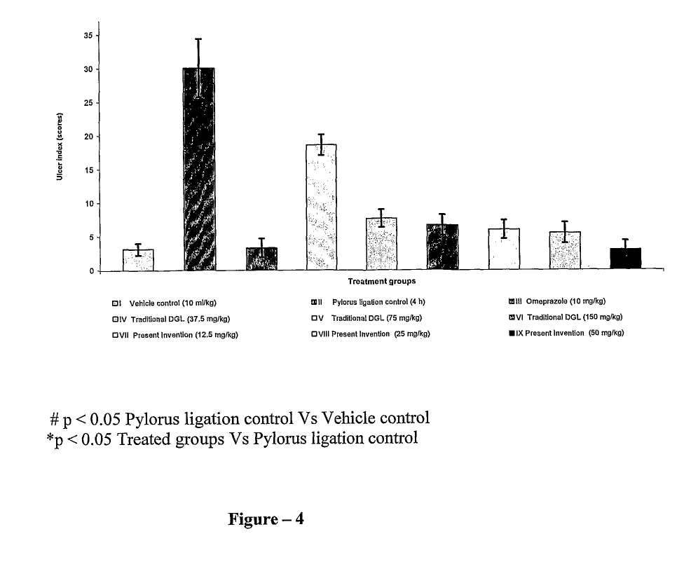

FIG. 5 Histogram showing results of pH values of treated groups in cold stress induced ulcer model using albino wistar rats

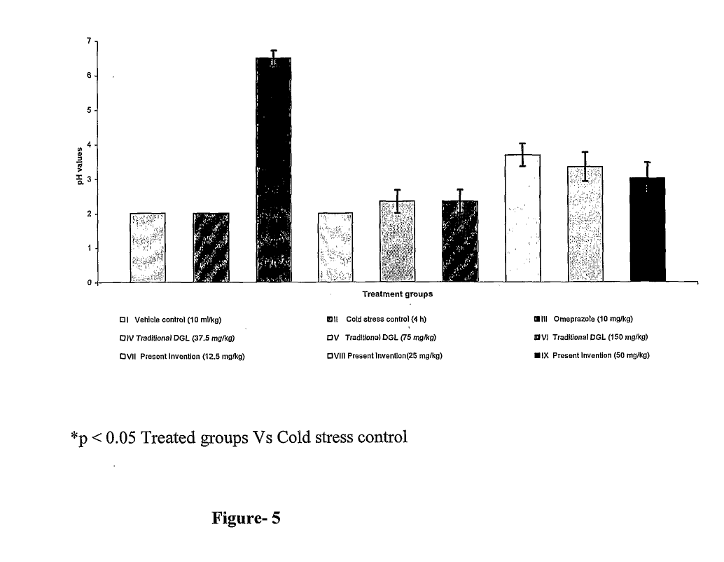

FIG. 6 Histogram of results of ulcer index of treated groups in cold stress induced ulcer model using albino wistar rats.

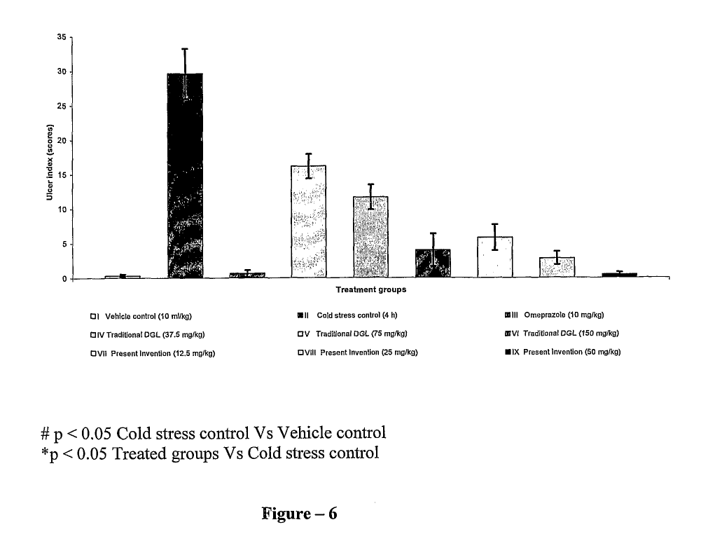

### DETAILED DESCRIPTION OF THE PRESENT INVENTION

The present invention is in relation to a phytochemical composition comprising eicosanyl caffeate and docosyl caffeate.

In one embodiment of the disclosure the composition as claimed in claim 1, wherein said composition is obtained from Glycyrrhiza species.

In one embodiment of the disclosure said eicosanyl caffeate and docosyl caffeate are in a ratio ranging from about 0.01 to 5% w/w.

In one embodiment of the disclosure said eicosanyl caffeate and docosyl caffeate are elastase inhibitors.

The present invention is in relation to a pharmaceutical composition comprising eicosanyl caffeate and docosyl caffeate optionally along with acceptable additives.

In one embodiment of the disclosure said composition optionally contains glabridin at a concentration ranging from about 1.0% w/w to about 3.5% w/w, flavonoids at a concentration ranging from about 5.0% w/w to 20.0% w/w and glycyrrhizinic acid and glycyrrhetinic acid at a concentration of about 0.3% w/w.

In one embodiment of the disclosure said composition can be formulated into dosage forms selected from a group comprising of tablet, troches, lozenges, aqueous or oily suspensions, dispersible powders or granules, emulsion in hard or soft gel capsules, syrups, elixirs.

The present invention is in relation to a process for preparation of a phytochemical composition comprising eicosanyl caffeate and docosyl caffeate, comprising steps of:
(a) extracting powdered plant parts with solvents to obtain extract;
(b) filtering, drying and fractionating the extract to obtain a residue; and
(c) subjecting the residue to preparative HPLC to obtain the synergistic composition.

In one embodiment of the disclosure said plant parts are from Glycyrrhiza species.

In one embodiment of the disclosure the plant parts are selected form a group comprising root, shoot, leaf and seeds or the whole plant.

In one embodiment of the disclosure the preferred plant parts are roots.

In one embodiment of the disclosure said extraction is successive extraction by refluxing or extraction by refluxing.

In one embodiment of the disclosure said successive extraction by refluxing is performed using solvents selected from a group comprising ethyl acetate, methanol and water.

In one embodiment of the disclosure said extraction by refluxing is performed using acetone.

The present invention is in relation to use of a phytochemical composition comprising eicosanyl caffeate and docosyl caffeate to manufacture a medicament for holistic management of ulcer, gut mucosal health and gut related disorders in a subject in need thereof.

In one embodiment of the present disclosure, said eicosanyl caffeate and docosyl caffeate are present in ratio ranging from about 0.01% to about 5%.

In one embodiment of the present disclosure, the subject is animal including human beings.

In one embodiment of the present disclosure the dose is ranging from about 100 mg to about 500 mg per day in divided doses or in a single dose.

Brief Description of G. glabra plant

Glycyrrhizins glabra (Fabaceae) commonly known as licorice, is one of the popular natural remedies useful in treating gastric damage (F. Borrelli, A. A. Izzo. Phytother. Res., 14, 581 (2000).) G. glabra has been reported to possess antioxidant activity in different in vitro assays and animal models (P. A. Bafna, R. Balaraman. Phytomedicine., 12, 264 (2005).)

Glycyrrhizin is a major bioactive compound present in Licorice and this compound has been attributed with many biological activities related to healing of ulcer like anti-inflammatory etc. Many flavonoid constituents have been reported from Licorice like isoflavans viz., glabridin, hispaglabridins A & B, 4′-O-methylglabridin; chalcones viz., isoliquiritgenin; isoflavones viz., formononetin [5-10]. (J. Vaya, P. A. Belinky, M. Aviram. Free Radic Biol Med, 23: 302 (1997)., H. Haraguchi, N. Yoshida, H. Ishikawa, Y. Tamura, K. Mizutani, T. Kinoshita. J. Pharm. Pharmacol., 52, 219 (2000)., T. Fukai, K. Satoh, T. Nomura, H. Sakagami. Fitoterapia., 74, 624 (2003a)., T. Fukai, K. Satoh, T. Nomura, H. Sakagami. Fitoterapia., 74, 720 (2003b)., P. A. Belinky, M. Aviram, B. Fuhrman, M. Rosenbat, J. Vaya. Atherosclerosis., 137, 49 (1998)., K. Okada, Y. Tamusa, M. Yamamoto, Y. Inou, R. Takagaki, K. Takahashi, S. Demizu, K. Kajiyama, Y. Hiraga, T. Kinoshita. Chem. Pharm. Bull., 37, 2528 (1989).

### Need of the Present Invention

Glycyrrhizin is a major bioactive compound present in Licorice and this compound has been attributed with many biological activities related to healing of ulcer like anti-inflammatory etc. However, Glycyrrhizin is supposed to be toxic at higher doses and may lead to hypokalemia and serious increases in blood pressure, a syndrome known as apparent mineralocorticoid excess. These side effects stem from the inhibition of the enzyme 11β-hydroxysteroid dehydrogenase (type 2) and subsequent increase in activity of cortisol on the kidney. 11β-hydroxysteroid dehydrogenase normally inactivates cortisol in the kidney; thus, liquorice's inhibition of this enzyme makes the concentration of cortisol appear to increase. Cortisol acts at the same receptor as the hormone aldosterone in the kidney and the effects mimic aldosterone excess, although aldosterone remains low or normal during liquorice overdose.

These concerns have somewhat been addressed in the deglycyrrhizinated licorice (DGL) preparations available in the market. These preparations are typically derived from water extracts of Licorice and removal of glycyrrhizin from the extract is achieved by acid precipitation of Glycyrrhizin from the water extract. DGL preparations contain less than 3% glycyrrhizin and hence are considered to be safe.

However, from efficacy point of view we observed some deficiencies in the traditional DGL preparations. These include high dose required to get minimum effect, lack of anti-oxidant activity, lack of elastase inhibitory activity, lack of anti-H pylori activity and lack of anti-inflammatory activity.

Overcoming these deficiencies of DGL preparations and providing a superior product has become a need and the present invention has been achieved while working on the need.

### Novelty of the Present Invention:

1. Inhibition of elastase known to contribute to healing of ulcers. This invention is about a composition containing two elastase inhibitory compounds from Licorice reported for the first time.
2. In addition the present composition has been shown to possess superior anti-ulcer activity in animal models when compared to traditional DGL extracts available in the market.
3. Traditional DGL extract does not contain (or contains negligible amounts) of two new compounds present in the invention.
4. In addition the said invention also possesses anti-oxidant, anti H. Pylori and anti inflammatory activity which is negligible in the traditional DGL preparations.

The technology of the instant Application is further elaborated with the help of following examples. However, the examples should not be construed to limit the scope of the invention.

### Example 1

The powdered plant material Glycyrrhiza roots (15 kg) were successively extracted with ethyl acetate (6 L×3), methanol (6L×3) and water (6L×3) by refluxing. The three extracts were separately filtered and dried under vacuum to yield 0.4 kg, 1.73 kg and 1.35 kgs from ethyl acetate, methanol and water, respectively. The ethyl acetate extract (300 g) was fractionated on a silica gel column using the following combinations of solvents: petroleum ether, ethyl acetate (95:5, 90:10, 80:20, 60:40, 25:75, 0:100); ethyl acetate, and methanol (95:5, 90:10, 75:25, 50:50, 0:100). The residue obtained from petroleum ether, ethyl acetate (8:2) fraction was further subjected to preparative HPLC to get eicosanyl caffeate (1) docosyl caffeate (2). The ¹H and ¹³C NMR data with complete results are provided in below table: 1.

### Example: 2

The powdered plant material Glycyrrhiza roots (15 kg) were extracted by refluxing with acetone (6 L×3) and the acetone extract was concentrated under vacuum at less than 50° C. to a thick paste. The resultant thick paste was dried in a vacuum tray dryer at less than 60° C. and the dried extract was milled and powdered to obtain the present invention which contains two caffeic acid derivative esters viz., eicosanyl caffeate (1) and docosyl caffeate (2).

TABLE 1
¹H and ¹³C NMR data of 1^{a} (δ in ppm, J in parenthesis in Hz)

| Carbon | δ_{H}^{b} | | δ_{H}^{c} | | δ_{c}^{c} | HMBC^{c} |
|---|---|---|---|---|---|---|
| 1 | 7.19 | (d, 1.9) | 7.63 | (s) | 128.1 | H-6, H-7, H-5, |
| | | | | | | H-8 |
| 2 | — | | — | | 116.4 | H-7 |
| 3 | — | | — | | 151.4 | H-2, H-5 |
| 4 | — | | — | | 148.8 | H-5, H-2, H-6 |
| 5 | 6.92 | (d, 8.2) | 7.19^{d} | | 117.8 | H-6 |
| 6 | 7.08 | (dd, 1.9, 8.2) | 7.19^{d} | | 123.0 | H-5, H-2, H-7 |
| 7 | 7.57 | (d, 15.99) | 8.02 | (d, 15.9) | 146.7 | H-8, H-2, H-6 |
| 8 | 6.31 | (d, 15.9) | 6.65 | (d, 15.9) | 116.9 | H-7 |
| 9 | — | | — | | 168.5 | H-8, H-1′, H-7 |
| 1′ | 4.18 | (t, 6.6) | 4.28 | (t, 6.7) | 65.5 | H-2′, H-8 |
| 2′ | 1.71 | (m) | 1.68 | (m) | 30.3 | H-1′ |
| 3′ | 1.33 | (brs)^{e} | 1.27 | (brs)^{e} | 27.4 | H-1′, H-2′ |
| 4′ | 1.33 | (brs)^{e} | 1.27 | (brs)^{e} | 30.6 | H-2′ |
| 5′-16′ | 1.33 | (brs)^{e} | 1.27 | (brs)^{e} | 31.0 | — |
| 17′ | 1.33 | (brs)^{e} | 1.27 | (brs)^{e} | 30.9 | — |
| 18′ | 1.33 | (brs)^{e} | 1.27 | (brs)^{e} | 33.2 | H-20′ |
| 19′ | 1.33 | (brs)^{e} | 1.27 | (brs)^{e} | 33.9 | H-20′ |
| 20′ | 0.91 | (t, 6.6) | 0.86 | (t, 6.7) | 15.2 | |
| OH-3 | 8.29 | (brs) | 11.59 | (brs) | — | — |
| OH-4 | 8.29 | (brs) | 12.07 | (brs) | — | — |

^{a}Assignments were confirmed with HSQC and DEPT experiments.
^{b}In acetone - d₆
^{c}In pyridine - d₅
^{d}Merged with solvent signals.
^{e}Area under the peak corresponded to 34-H

### Example 3

### Elastase Inhibition Assay [11]

In brief, 233 μl of each test solutions/reference standard (various concentrations) in 100 mM Tris-HCl pH8.0 and 7 μl of enzyme (porcine pancreatic elastase, 0.84 units/ml) solution was incubated at 37° C. for 15 mins. Following incubation, 20 μl of substrate (n-succinyl-ala-ala-ala-p-nitroanilide) solution was added and incubated at 37° C. for 30 mins. The absorbance was measured at 405 nm. A control reaction was carried out without the test sample. The percentage inhibition was calculated by the equation [absorbance (control)−absorbance (test)]/absorbance (control)×100]. The IC₅₀ values were determined using log-probit analysis. The results are provided in table 2.

TABLE 2
IC₅₀ of isolates of G. glabra in elastase inhibition assay

| Sample | IC₅₀^{a} | 95% C.I |
|---|---|---|
| 1 Eicosanyl caffeate(1) | 0.99 | 0.8-1.1 |
| 2 Docosyl caffeate (2) | 1.4 | 1.2-1.5 |
| Ursolic acid (positive control) | 10.8 | 8.8-13.5 |

^{a}Expressed in μg/ml
C.I Confidence interval

### Example 4

### DPPH Scavenging Assay [12]

A final reaction volume of 250 μl in methanol contained different concentrations of the tested samples (two caffeic acid derivative esters viz., eicosanyl caffeate (1) docosyl caffeate (2)) and gallic acid. The final concentrations tested were 0.5 to 100 μg/ml for extracts, 1.0 to 30 m/ml for eicosanyl caffeate 1 and docosyl caffeate 2. 0.5 to 2.5 μg/ml for gallic acid and 10 μl of DPPH solution (1.3 mg/ml in methanol). The tubes were mixed thoroughly, incubated at 25° C. for 15 min and the absorbance was measured at 510 nm. A control reaction was carried out without the test sample. Percentage inhibition was derived from the equation: [absorbance (control)−absorbance (test)]/absorbance (control×100) and the IC₅₀ values were calculated by log−probit analysis. The results are provided in Table 3.

TABLE 3
IC₅₀ of fractions and isolates of G. glabra in
DPPH scavenging & ABTS radical scavenging assay

| | DPPH | | ABTS | |
|---|---|---|---|---|
| Sample | IC₅₀^{a} | 95% C.I | IC₅₀^{a} | 95% C.I |
| Ethyl acetate extract | 20.1 | 13.2-32.5 | ND | ND |
| Successive Licorice | NA^{b} | — | ND | ND |
| methanol extract | | | | |
| Successive licorice | NA^{b} | — | ND | ND |
| water extract | | | | |
| 1 Eicosanyl caffeate(1) | 8.8 | 6.8-12.6 | 20.3 | 17.9-23.5 |
| 2 Docosyl caffeate (2) | 13.2 | 11.4-15.6 | 23.1 | 19.6-27.6 |
| Gallic acid (positive control) | 0.8 | 0.6-0.9 | 1.4 | 1.2-1.6 |

^{a}Expressed in μg/ml
^{b}No significant inhibition observed up to 100 μg/ml
C.I Confidence interval
ND Not determined

### Example 5

### ABTS Radical Scavenging Assay [13]

20 μl of each test solutions namely two caffeic acid derivative esters viz., eicosanyl caffeate (1) docosyl caffeate (2)/reference standard of various concentration/Phosphate buffer saline (PBS) and 230 μl of ABTS (0.238 mm) solution were mixed. The absorbance was measured immediately at 734 nm. The percentage inhibition was calculated by the equation [absorbance (control)−absorbance (test)]/absorbance (control)×100]. The IC₅₀ values were determined using log-probit analysis. The results are provided in Table 3 as above.

### Example 6

### Anti Ulcer Potential of Present Invention in Pylorus Ligation Induced Ulcer Model

This study was undertaken to evaluate the anti ulcer activity of traditional

Deglycyrrhizinated Licorice (DGL) and present invention using pylorus ligation induced ulcer model in albino Wistar rats. Details of the study are provided in the below table: 4.

TABLE 4
details of the pylorus ligation study

| | |
|---|---|
| Title: | Anti ulcer potential of present invention in pylorus ligation |
| | induced ulcer model |
| Animals | Albino Wistar rats (either sex). |
| Test Material | Present invention; Traditional DGL and Omeprazole |
| | (positive control) |
| Dose & | Present invention at (2.5, 25, 50 mg/kg), Omeprazole at |
| duration | 10 mg/kg and Traditional DGL at (37.5, 75, 150 mg/kg) |
| Results | Present invention showed a significant anti-ulcer activity |
| | in pylorus ligation induced ulcer model in a dose dependent |
| | manner. As compared to effect seen at 12.5 mg/kg with |
| | the present invention, Traditional DGL showed activity at |
| | 150 mg/kg only. |

TABLE 5
Observations of treated groups in Pylorus ligation
induced ulcer model using albino wistar rats

| | Gastric | | Total acidity | |
|---|---|---|---|---|
| Treatment group | content (ml) | pH | (mEq/L/100 g) | Ulcer index |
| I | 0.00 ± 0.00 | 2.00 ± 0.00 | 0.00 ± 0.00 | 3.17 ± 0.87 |
| Vehicle control | | | | |
| (10 ml/kg) | | | | |
| II | 6.33 ± 0.31^{#} | 2.15 ± 0.06 | 120.67 ± 5.56^{#} | 30.00 ± 4.33^{#} |
| Pylorus ligation control | | | | |
| (4 h) | | | | |
| III | 2.42 ± 0.24* | 6.89 ± 0.14* | 15.33 ± 0.92* | 3.33 ± 1.38* |
| Omeprazole | | | | |
| (10 mg/kg) | | | | |
| IV | 5.42 ± 0.35 | 2.20 ± 0.07 | 113.33 ± 3.00 | 18.67 ± 1.54 |
| Traditional DGL | | | | |
| (37.5 mg/kg) | | | | |
| V | 4.58 ± 0.51 | 2.34 ± 0.13 | 103.00 ± 9.37 | 7.67 ± 1.31 |
| Traditional DGL | | | | |
| (75 mg/kg) | | | | |
| VI | 4.83 ± 0.33 | 2.39 ± 0.08 | 83.67 ± 8.98 | 6.67 ± 1.52 |
| Traditional DGL | | | | |
| (150 mg/kg) | | | | |
| VII | 3.83 ± 0.28* | 2.45 ± 0.14 | 92.33 ± 5.40 | 6.00 ± 1.37* |
| Present invention. | | | | |
| (12.5 mg/kg) | | | | |
| VIII | 3.58 ± 0.35* | 2.55 ± 0.17 | 84.83 ± 8.34 | 5.50 ± 1.54* |
| Present invention | | | | |
| (25 mg/kg) | | | | |
| IX | 3.33 ± 0.33* | 2.72 ± 0.22 | 57.50 ± 4.89* | 3.00 ± 1.37* |
| Present invention. | | | | |
| (50 mg/kg) | | | | |

Values are expressed as mean ± SEM; n = 6
^{#}p ≦ 0.05 Pylorus ligation control Vs Vehicle control.
*p ≦ 0.05 Treated groups Vs Pylorus ligation control.

### Example 7

Anti ulcer potential of present invention in cold stress induced ulcer model Objective: The present study was undertaken to evaluate the anti ulcer activity of Traditional Deglycyrrhizinated Licorice (DGL) and present invention in cold stress induced ulcer model using albino Wistar rats. Details of the present study are provided in Table: 8.

TABLE 8
Details of the present study - Cold stress induced ulcer model

| | |
|---|---|
| Title: | Anti ulcer potential of present invention in cold stress |
| | induced ulcer model |
| Animals | Albino Wistar rats (either sex). |
| Test Material | Present invention; Traditional DGL and Omeprazole |
| | (positive control) |
| Dose & | Present invention (12.5, 25, 50 mg/kg), Traditional |
| duration | DGL at (37.5, 75, 150 mg/kg) and Omeprazole |
| | (10 mg/kg). |
| Results | Present invention showed a significant anti-ulcer |
| | activity in pylorus ligation induced ulcer model in a |
| | dose dependent manner. As compared to effect seen at |
| | 12.5 mg/kg with the present invention, Traditional DGL |
| | showed activity at 150 mg/kg only. |

TABLE 9
Observations of treated groups in cold stress
induced ulcer model using albino Wistar rats

| Treatment group | pH | Ulcer index |
|---|---|---|
| I | 2.00 ± 0.00 | 0.33 ± 0.21 |
| Vehicle control | | |
| (10 ml/kg) | | |
| II | 2.00 ± 0.00 | 29.67 ± 3.62^{#} |
| Cold stress control | | |
| (4 h) | | |
| III | 6.50 ± 0.22* | 0.67 ± 0.49* |
| Omeprazole | | |
| (10 mg/kg) | | |
| IV | 2.00 ± 0.00 | 16.17 ± 1.78 |
| Traditional DGL | | |
| (37.5 mg/kg) | | |
| V | 2.33 ± 0.33 | 11.67 ± 1.80 |
| Traditional DGL | | |
| (75 mg/kg) | | |
| VI | 2.33 ± 0.33 | 4.00 ± 2.35* |
| Traditional DGL | | |
| (150 mg/kg) | | |
| VII | 3.67 ± 0.33 | 5.83 ± 1.87* |
| Present invention. | | |
| (12.5 mg/kg) | | |
| VIII | 3.33 ± 0.42 | 2.83 ± 0.95* |
| Present invention. | | |
| (25 mg/kg) | | |
| IX | 3.00 ± 0.45 | 0.50 ± 0.34* |
| Present invention. | | |
| (50 mg/kg) | | |

Values are expressed as mean ± SEM; n = 6
^{#}p ≦ 0.05 Cold stress control Vs Vehicle control.
*p ≦ 0.05 Treated groups Vs Cold stress control.

### Example 8

### Anti-H. pylori Potential of Present Invention

Objective: To evaluate the anti H. pylori activity of the present invention
Samples: Sample #1: Traditional DGL-DGL/07/Lot 05
   Sample #2: Present invention B. No. PC/GG/AE/06
      Models & Protocols: Anti Helicobacter pylori Activity

### (i) Disc Diffusion Sensitivity Assay

(ii) MIC/MBC study: Microbroth Dilution Assay
Anti Helicobacter pylori Activity—Disc Diffusion Sensitivity Assay
Assays were performed employing one clinical (80A) and one standard (ATCC 43504) strain (Glupczynski, 1996).
Strains were maintained and cultured under appropriate growth condition.
Plates were kept for appropriate time in Incubator under optimum growth condition.
Inhibition zone diameter was measured after confluent growth.
Both the samples were screened employing 100, 200, 400 and 800 μg/disc dose.
Clarithromycin was employed as standard drug using 10 ng/disc for the strains 80A and 40 ng/disc for ATCC 43504.
For methanol containing sample, discs impregnated with samples were dried inside the hood and then placed on the plate. For water containing sample, discs were first placed on the plate and then 5-10 μl of each sample, was added. The results are shown in below table: 10.

TABLE 10
Disc diffusion studies on product of present invention

| | | | | Inhibition Zone | |
|---|---|---|---|---|---|
| | | | | Diameter (cm) in | |
| | | | | strain | |
| | | | Dose | | ATCC |
| Sample | Solvent | Solubility | (μg/disc) | 80A | 43504 |
| Traditional DGL | Water | Partly | 100 | Nil | Nil |
| Sample # 1 | | Soluble | 200 | 0.6 | Nil |
| (DGL/07/Lot 05) | | | 400 | 0.6 | Nil |
| | | | 800 | 0.6 | Nil |
| Present invention | Methanol | Slightly | 100 | 0.9 | 0.9 |
| Sample # 2 | | Insoluble | 200 | 1.0 | 1.1 |
| (B. No. | | | 400 | 1.4 | 1.1 |
| PC/GG/AE/06) | | | 800 | 1.5 | 1.2 |

| | |
|---|---|
| Clarithromycin | 1.8-2.0 |

### Observation:

Sample 1 did not show any anti Helicobacter pylori activity.
Sample #2 i.e, present invention showed activity.
Clarithromycin served as internal control for the assay, and showed appropriate zone of inhibition under the experimental condition.

### Minimum Inhibitory Concentrations (MICs): Microbroth Dilution Assay

MICs of the two samples were determined employing one clinical (80A) and one standard (ATCC 43504) strain (Osato, 2000).
Two-fold serial dilutions of the samples were performed in 96-well microtitre plate.
Plates were kept for appropriate time in Incubator under optimum growth condition.
Following incubation, the plates were examined visually, and the lowest concentration showing complete inhibition of growth was recorded as the MIC of the respective sample.

### Minimum Bactericidal Concentrations (MBCs)

Aliquots of culture in which no growth had been detected were taken from the wells of the above microtitre plates and used to streak on fresh agar plates.
MBCs were determined by visual inspection of such plates after further incubation and the point where no growth (less than 10 colony) appeared was considered as the MBCs.

TABLE 11
MIC and MBC Values

| | MIC (μg/ml) | | MBC (μg/ml) | |
|---|---|---|---|---|
| Sample | 80A | 43504 | 80A | 43504 |
| Traditional DGL | >800 | >800 | ND | ND |
| Sample # 1 | | | | |
| (DGL/07/Lot 05) | | | | |
| Present invention | 100 | 100 | 200 | 200 |
| Sample # 2 | | | | |
| (B. No. PC/GG/AE/06) | | | | |
| Clarithromycin | 0.013 | 0.025 | 0.025 | 0.05 |

Cell count: ~10⁶-10⁷ CFU/ml

### Observation:

Sample #1, showed negligible bacteriostatic and bactericidal activity against H. pylori.
Only Sample #2 i.e, Present invention showed moderate bacteriostatic and bactericidal activity against both the strains at 100 and 200 μg/ml respectively.

SAFETY STUDIES: A safety study was conducted to determine the acute oral toxicity of Present invention on Sprague Dawley rats and it is found to be safe up to 5 gms/kg rat body weigh. Table: 12 shows advantages of present invention vis-à-vis traditional compositions:

TABLE 12
Advantages of present invention vis-à-vis the traditional compositions

| Sl | | Traditional | Present | |
|---|---|---|---|---|
| No. | Points | DGL | invention | Remarks |
| 1 | Dose (human) | Typically: | 150 mg-250 | Dose of present |
| | | 1500 to 3000 | mg per day | invention is about 6 |
| | | mg per day | | to 10 times less. |
| 2 | Glycyrrhizin | 2-3% | Nil or less | No glycyrrhizin is |
| | | | than 0.2% | considered |
| | | | | advantageous (USP) |
| 3 | Process | Employs acid | No acid | No acid makes it |
| | | | | interesting and a USP |
| 4 | Antioxidant | Negligible | Very high | Antioxidant activity is |
| | activity | | | considered important |
| | | | | for ulcer healing |
| 5 | Taste | Bitter | Bland | Clear advantage of |
| | | | | present invention is |
| | | | | seen w.r.t. taste |
| 6 | In vitro activity | Poor | Medium to | Original data available |
| | against | | good | |
| | Helicobacter pylori | | | |
| 7 | In vivo animal | Effective at | Effective at | present invention |
| | studies (cold | 150 mg/kg | 12.5 mg/kg | works almost at 8-10 |
| | stress induced | (UI: 4.0) | (UI: 5.83) | times lesser dose than |
| | ulcer in rats) | | | regular DGL |
| 8 | Effect on pH of | Not | Not | Omeprazole increased |
| | stomach | significant | significant | the pH to more than 6 |
| 9 | In vivo animal | Effect not | Effective at | Same as above |
| | studies (pylorus | statistically | 12.5 mg/kg | |
| | ligation induced | significant at | (UI: 6.0) | |
| | ulcer) | 150 mg/kg | | |

1-17. (canceled)

18. A composition comprising eicosanyl caffeate and docosyl caffeate optionally along with acceptable additives.

19. The composition as claimed in claim 18, wherein said composition is obtained from Glycyrrhiza species.

20. The composition as claimed in claim 18, wherein said eicosanyl caffeate and docosyl caffeate are in a ratio ranging from about 0.01 to about 5% w/w.

21. The composition as claimed in claim 18, wherein said eicosanyl caffeate and docosyl caffeate are elastase inhibitors.

22. The composition as claimed in claim 18, wherein said composition further comprises glabridin at a concentration ranging from about 1.0% w/w to about 3.5% w/w, flavonoids at a concentration ranging from about 5.0% w/w to about 20.0% w/w and glycyrrhizinic acid and glycyrrhetinic acid at a concentration of about 0.3% w/w.

23. The composition as claimed in claim 18, wherein said composition is formulated into dosage forms selected from a group comprising tablet, troches, lozenges, aqueous suspensions, oily suspensions, dispersible powders, dispersible granules, emulsion in hard gel capsules, soft gel capsules, syrups and elixirs.

24. A process for preparation of a composition comprising eicosanyl caffeate and docosyl caffeate, said process comprising steps of:
(a) extracting powdered plant parts of Glycyrrhiza species with water or organic solvent to obtain an extract;
(b) filtering, drying and fractionating the extract with combination of solvents to obtain a residue; and
(c) subjecting the residue to preparative HPLC to obtain the composition; and
(d) optionally, adding acceptable additives.

25. The process as claimed in claim 24, wherein said composition further comprises glabridin at a concentration ranging from about 1.0% w/w to about 3.5% w/w, flavonoids at a concentration ranging from about 5.0% w/w to about 20.0% w/w and glycyrrhizinic acid and glycyrrhetinic acid at a concentration of about 0.3% w/w.

26. The process as claimed in claim 24, wherein the plant parts are selected from a group comprising root, shoot, leaf, seeds and the whole plant, preferably the roots.

27. The process as claimed in claim 24, wherein said extraction is by refluxing.

28. The process as claimed in claim 24, wherein the organic solvent in step (a) is selected from a group comprising acetone, ethyl acetate and methanol; and wherein the combination of solvents is selected from a group comprising (a) petroleum ether and ethyl acetate; and (b) ethyl acetate and methanol.

29. A process for treating an ulcer in a subject in need there of, the process comprising: administering a composition comprising eicosanyl caffeate and docosyl caffeate, optionally along with acceptable additives to manufacture a medicament.

30. The method as claimed in claim 29, wherein said composition shows inhibition of elastase and Anti Helicobacter pylori activity.

31. The method as claimed in claim 29, wherein said eicosanyl caffeate and docosyl caffeate are present in ratio ranging from about 0.01% to about 5%.

32. The method as claimed in claim 29, wherein the subject is an animal including human beings; and wherein the medicament is administered at a dosage ranging from about 100 mg to about 500 mg per day in divided doses or in a single dose.
